(19) United States
(12) Patent Application Publication
(10) Pub. No.: US 2021/0205603 A1
(43) Pub. Date: Jul. 8, 2021

(54) DEVICE FOR ANCHORING A HEART PUMP

(71) Applicant: FINEHEART, Pessac (FR)

(72) Inventors: Jérémy COLLAS, Bordeaux (FR); Jean-Baptiste ROUSSEAU, Bordeaux (FR); Arnaud MASCARELL, Montbazon (FR); Stéphane GARRIGUE, Begles (FR)

(21) Appl. No.: 17/057,205

(22) PCT Filed: May 22, 2019
(86) PCT No.: PCT/FR2019/051162

§ 371 Date: Nov. 20, 2020

(30) Foreign Application Priority Data
May 22, 2018 (FR) 18 54245

(51) Int. Cl. A61M 60/863; A61M 60/174; A61M 60/859; A61M 60/894

(52) U.S. Cl. CPC A61M 60/863; A61M 60/894; A61M 60/859; A61M 60/174

(57) ABSTRACT

A medical device, for anchoring a heart pump in an opening of a ventricular wall of a heart, includes a first flange having a hole, an assembly having a hollow body, at the distal end of which is placed a membrane which is self-expandable between a first configuration in which said membrane has a tubular shape, and a second configuration in which it defines a second flange, the first flange being separate from the assembly, the assembly being designed to be at least partially introduced through the hole of the first flange, and a clamping means for connecting the first flange and the proximal end of said hollow body when this assembly has been partially inserted into the hole, the clamping means being configured to slide along the hollow body in such a way as to be pressed against the first flange.

### CROSS REFERENCE TO RELATED APPLICATIONS

This application is a National Stage of International Application No. PCT/FR2019/051162, having an International Filing Date of 22 May 2019, which designated the United States of America, and which International Application was published under PCT Article 21(2) as WO Publication No. 2019/224478 A1, which claims priority from and the benefit of French Patent Application No. 1854245, filed on 22 May 2018, the disclosures of which are incorporated herein by reference in their entireties.

### BACKGROUND

### 1. Field

The present disclosure relates to a device for anchoring a heart pump in an opening of a ventricular wall of a beating heart. It also relates to a method for placing this anchoring device on such a wall.

### 2. Brief Description of Related Developments

Heart failure (HF) is a pathological state in which the heart of a patient is unable to provide a rate of blood flow necessary for the metabolic needs of the body.

It is known to treat heart failure by implanting a ventricular assist device (VAD), which is an artificial heart pump.

This mechanical pump does not replace the heart, which continues to function, but helps the weakened ventricle to increase the rate of blood flow in order to meet the needs of the individual.

This assistance may be temporary while waiting for a graft to become available for a heart transplant.

However, a significant proportion of patients will not receive such a transplant, either because they may not be candidates for such a transplant, for example because of severe heart failure, or because no suitable graft is available for these patients.

In that case, ventricular assistance is used as the intended solution, which is to say that the artificial heart pump is implanted for the long term.

These heart pumps are therefore the subject of intensive research aimed at improving the survival and the quality of life of patients presenting with heart failure.

Many advances have been made in recent years, and today we have ventricular assist devices which are more compact and quieter and which provide an increased service life.

The implantable heart pumps of the prior art are thus typically equipped with an integrated electric motor to ensure their operation, the speed of rotation of the pump providing the force necessary for circulating the blood from the weakened ventricle to the circulatory system.

Systems for implanting such pumps in an opening of a ventricular wall are known.

These implantation systems generally comprise a tubular portion, at the ends of which are placed, or formed, collars which are intended to be each pressed against an opposite face of the ventricular wall after insertion of the tubular portion into an opening made in this ventricular wall with a coring device.

These collars thus make it possible to maintain this hollow tubular portion in position, which then defines an open conduit passing through the ventricular wall.

At the end, placed outside the heart, of this tubular portion, a heart pump is inserted which, once fitted, ensures that the blood present in the ventricle returns to the circulatory system.

Although representing some progress in terms of the quality of life of a patient suffering from heart failure, there are still many disadvantages.

In particular, forming this opening on a beating heart in order to minimize the side effects of the intervention on the patient can lead to significant blood loss during the implantation of the heart pump.

Important factors for achieving a successful outcome therefore include great dexterity and speed of action on the part of the practitioners, all of which places a strain on the team performing the intervention.

In addition, it is desirable that the blood ejection end of the pump is centered on the cardiac valve in order to ensure an optimal flow of ejected blood.

The positioning of the opening in the ventricular wall thus influences the actual performance of the heart pump in ejecting blood into the aorta.

However, in the absence of markers, it has been found that this opening is not always well positioned with respect to the cardiac valve. Consequently, the flow of blood is not optimally ejected.

There is therefore an urgent need for an anchoring device whose novel design overcomes the disadvantages described above.

### SUMMARY

The present disclosure aims to overcome the drawbacks of the prior art and to meet the above demands by making available a device for anchoring a heart pump and its method of placement, which are simple in terms of their design and operating mode and are reliable and ensure total leaktightness between the inside and outside of the heart.

Another object of the present disclosure is to make available such an anchoring device and such a method which reduce the complexity of the surgical intervention.

Another object of the present disclosure is to make available such an anchoring device and such a method which are able to reduce the blood loss suffered by the patient during the surgical intervention and improve his or her recovery.

To this end, the disclosure relates to a method for placing a device for anchoring a heart pump on a heart.

According to the disclosure with said anchoring device comprising a hollow body at the proximal end of which is intended to be placed a first attached flange and at the distal end of which is placed a membrane self-expandable between a first configuration, called a deformed configuration, in which said membrane has a tubular or substantially tubular shape, and a second configuration, called an initial configuration, in which it defines a second flange, the following steps are carried out:

a) said first flange being separate from the assembly composed of hollow body and self-expanding membrane, said first flange is first of all connected to the outer surface of the ventricular wall of said heart,

b) with said first flange having a hole, preferably a central hole, said first flange is arranged such that said hole communicates with an opening in the ventricular wall when said opening already exists, or an opening is made in said ventricular wall through said hole of said first flange thus assembled, when this opening is not yet formed,

c) the assembly comprising said hollow body and the self-expanding membrane in its deformed configuration is introduced through said hole of said first flange and said opening in the ventricular wall,

d) with said self-expanding membrane being placed in the ventricular chamber and assuming its initial configuration, said second flange is pressed against an inner surface of said ventricular wall, said hollow body being configured such that its proximal end then protrudes from said first flange outside said heart,

e) said first flange and said hollow body are connected in a leaktight manner such that, with said flanges being pressed on either side of said ventricular wall, said anchoring device is held, or locked, in position.

Such a device for anchoring a heart pump in the opening of a ventricular wall of a beating heart is also known as an anchoring ring, or fastening device, of a heart pump.

Advantageously, such an anchoring device provides increased convenience for the surgical team during the intervention, since this anchoring device is easier to fit in place. It also provides enhanced safety for the patient, since a total seal is achieved between the inside and outside of the patient's heart.

Advantageously, the self-expanding membrane has a first configuration, called a deformed configuration, in which it has a tubular or substantially tubular shape, and a second configuration, called an initial configuration, in which it defines a flange, or collar, extending radially, or substantially radially, from said hollow body, said flange being intended to come into abutment against an inner face of said ventricular wall of the beating heart.

Preferably, the assembly composed of hollow body and self-expanding membrane has a sealing element comprising an anti-reflux valve which, for example upon withdrawal of the heart pump received in the hollow body, closes and becomes leaktight again. By way of example, it is a self-repairing membrane, also called a self-sealing membrane, integrated in the internal channel delimited by the hollow body of the anchoring ring. This sealing element advantageously prevents any leakage of blood through this internal channel.

In various particular embodiments of this placement method, each having its particular advantages and susceptible of many possible technical combinations:

in step a), said first flange is connected by sewing to the outer surface of said ventricular wall.

This step may, for example, involve the formation of several surgical suture points.

in step a), said first flange is connected to the outer surface of said ventricular wall by suction.

Preferably, with this first flange having two end faces connected to each other by a side wall and said end face being intended to come into abutment against the outer surface of said ventricular wall, comprising a groove in communication with an orifice opening out laterally, said end face is pressed against said outer surface and a vacuum is created by suction in order to connect said first flange and said ventricular wall.

1. in step a), with said opening already being present in said ventricular wall, the hole of said first flange is centered on said opening before connection to said ventricular wall. This ensures optimization of the dimensions of the path created by the hole of said first flange and the opening of said ventricular wall. Of course, when the opening in the ventricular wall already exists, the hole of said first flange is arranged such that it communicates, and better still communicates fully, with this opening before connection of this first flange to said ventricular wall.

2. prior to step a), an incision is made in the ventricular wall, and a guidewire is introduced into the ventricular chamber of the heart and through the corresponding cardiac valve in order to position said first flange. This wire will not only guide the first flange into position but is also intended for guiding, in particular for aligning, the coring head as it approaches the ventricular wall with a view to producing an opening by coring in this wall, when an opening is not already formed.

3. in step e), said first flange and the proximal end of said hollow body are connected by a clamping means such as a nut or a clamping ring. Preferably, this clamping means will bear against the other end face of the first flange. Advantageously, a sealing element is placed between this clamping means and this first flange, for example an O-ring seal.

4. a heart pump is introduced into the internal channel defined by the hollow body of the anchoring device, and this pump is connected to the hollow body. Preferably, this connection is a temporary or reversible coupling, such that the heart pump can be easily withdrawn during a handling step.

the various steps of this method are performed on a beating heart.

The present disclosure also relates to a medical device for anchoring a heart pump in an opening of a ventricular wall of a heart, comprising:

a first flange comprising a hole, preferably a central hole,

an assembly having a hollow body, at the distal end of which is placed a membrane which is self-expandable between a first configuration, called a deformed configuration, in which said membrane has a tubular shape, and a second configuration, called an initial configuration, in which it defines a second flange,

said first flange being separate from said assembly, said assembly being configured to be at least partially introduced through the hole of said first flange,

a clamping means for connecting said first flange and the proximal end of said hollow body when this assembly has been partially inserted into the hole of said first flange, said clamping means being configured to slide along said hollow body in such a way as to be pressed against said first flange.

It will be clear that the clamping means surrounds the outer surface of said hollow body when it is engaged on the latter.

Advantageously, this medical anchoring device is made entirely of a biocompatible, non-toxic and sterile material.

According to one embodiment of the medical anchoring device of the invention, the clamping means is configured to effect a reversible or temporary coupling of said first flange and the proximal end of said hollow body.

“Reversible” means that said first flange and said hollow body, after they have been connected, can be disconnected from each other while remaining functional for a new subsequent connection.

According to another embodiment of the anchoring device of the disclosure, this device also comprises a first sealing element intended to be placed between said clamping means and said first flange.

This sealing element is advantageously an O-ring seal.

Advantageously, the clamping means can comprise a housing for receiving this first sealing element, the latter protruding from the clamping means when placed in its housing.

According to yet another embodiment of this anchoring device, it has a second sealing element comprising an anti-reflux valve placed in the internal channel delimited by said hollow body.

By way of example, it is a self-repairing membrane, also called a self-sealing membrane, integrated in the internal channel delimited by the hollow body of the anchoring ring. This anti-reflux valve prevents any leakage of blood through this internal channel. After withdrawal of the coring device, it thus makes it possible to restore the leaktightness when the anchoring device is placed on the heart. Upon removal of the heart pump from the heart, for example for handling, received in the hollow body, this second sealing element closes and becomes leaktight again.

Purely by way of illustration, this anti-reflux valve is made of silicone.

According to yet another embodiment of this anchoring device, this first flange is made of a material that can be sutured or stapled.

Purely by way of illustration, this first flange is made of Dacron®.

Alternatively, this first flange is configured to be connected to said ventricular wall by creation of a negative pressure between said first flange and said ventricular wall.

By way of example, this first flange having two end faces connected to each other by a side wall, its end face intended to come into abutment against the outer surface of said ventricular wall, comprises a circular groove in communication with an orifice opening out on its side wall.

According to yet another embodiment of this anchoring device, this hollow body has a length greater than the sum of the thickness of the ventricular wall and said first flange.

According to yet another embodiment of this anchoring device, the clamping means is a nut.

With this nut having an internal thread, the outer surface of a part of the hollow body, in particular its proximal end, has a complementary thread matching the thread of the nut in order to ensure the engagement and the movement of the nut along said outer surface part of said hollow body.

According to yet another embodiment of this anchoring device, the proximal end of the hollow body has a bore for the screwing-in or passage of a fastening element such as a screw, so as to lock in position a heart pump that is introduced into this hollow body.

According to yet another embodiment of this anchoring device, said self-expanding membrane is made of Nitinol.

### BRIEF DESCRIPTION OF THE DRAWINGS

Other advantages, aims and particular features of the present disclosure will become clear from the following description, which is provided for explanatory purposes and is non-limiting and in which reference is made to the accompanying drawings, in which:

FIG. 1 shows schematically a coring device for making an opening in a ventricular wall of a beating heart, according to a particular embodiment of the present disclosure;

FIG. 2 shows the coring device from FIG. 1, an accessory for assistance in the placement of an anchoring ring being received at the distal end thereof in order to define an assembly for coring and for inserting a part of an anchoring ring of a ventricular assist device;

FIG. 3 is a schematic representation of the assembly from FIG. 2, the coring head being in its actuated position for its insertion into a small orifice, previously made with a cutting tool, in the ventricular wall of the heart;

FIG. 4 is a perspective and sectional view of the assembly from FIG. 2, just after the cut has been made in the ventricular wall;

FIG. 5 is a partial top view of the assembly from FIG. 2, during the connection of the assistance accessory to the flange previously connected to the outer surface of said ventricular wall, the distal end of the assistance accessory then being inserted into the opening that is formed;

FIG. 6 is a longitudinal sectional view of the assembly from FIG. 2 and of the apex of the heart, the coring device being in the course of being withdrawn, the distal end of the assistance accessory, inserted in the formed opening, being exposed;

FIG. 7 is a perspective view of the assistance accessory from FIG. 2 connected to a flange rigidly connected to the apex of the heart, a part of an anchoring ring having a flange in its deformed configuration being inserted into this assistance accessory by means of a pusher device;

FIG. 8 is a longitudinal sectional view of the elements illustrated in FIG. 7;

FIG. 9 is a longitudinal sectional view of the elements illustrated in FIG. 7, the flange in its deformed configuration being inserted into the ventricular chamber by means of the pusher device;

FIG. 10 is a longitudinal sectional view of the elements illustrated in FIG. 7, the flange being in its initial configuration in order to define a collar, the placement assistance accessory having been disconnected from the flange mounted on the outer surface of the ventricular wall and being in the course of being withdrawn;

FIG. 11 is a perspective view of the elements illustrated in FIG. 7, the assistance accessory having been withdrawn, a seal and a nut being engaged on the pusher device in order to lock the anchoring ring in position;

FIG. 12 is a perspective view of the anchoring ring from FIG. 11 mounted in its opening and supporting a heart pump.

### DETAILED DESCRIPTION

Firstly, it will be noted that the figures are not to scale.

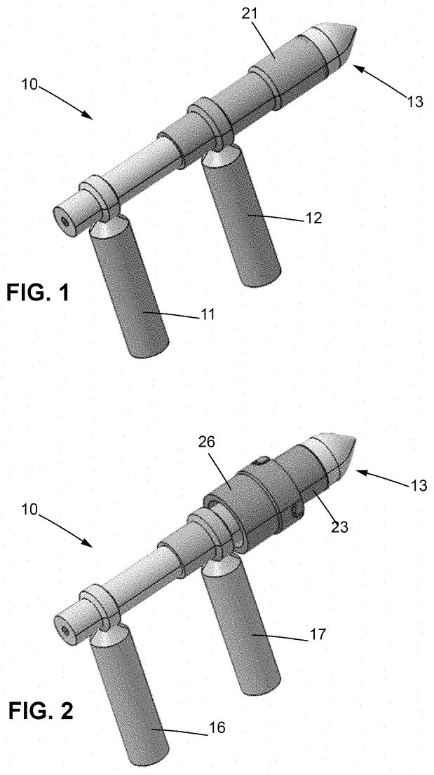

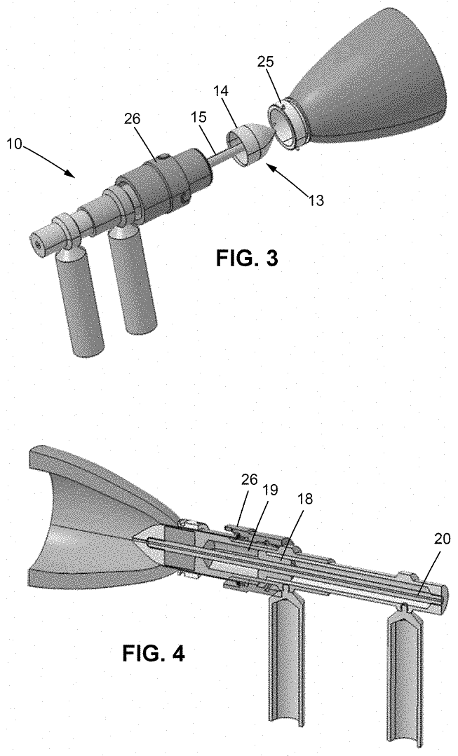

FIGS. 1 to 6 show schematically a coring device 10 for making an opening in the ventricular wall of a beating heart, according to a particular embodiment of the present disclosure.

This coring device 10 has two body parts 11, 12 which are engaged one inside the other in such a way that one of these parts is mounted slidably in the other body part. This device has a proximal end and a distal end.

In the context of the present disclosure, the term “proximal” means the position closest to the healthcare professional or practitioner, while the term “distal” must be understood here as meaning farthest away from this professional. In other words, the distal end of a component is the end that would be engaged first in the beating heart, while the proximal end of said component would be the last end to be engaged therein.

This coring device 10 comprises a coring head 13 having a tip with a frustoconical shape, at its distal end, in order to facilitate its insertion through an incision, made with a cutting tool such as a scalpel, in the ventricular wall of this beating heart. This head 13 also has a transection blade 14 placed at its proximal end. This transection blade 14 has a straight cross section of circular shape, although it could have other appropriate shapes, for example oval.

This coring head 13 is movable between a rest position, in which the transection blade 14 is pressed against, or is placed near, the distal end of the main body of the coring device 10, and an actuated position, in which the transection blade 14 is placed at a distance from the distal end of the main body of the coring device 10.

More precisely, this coring head 13 is actuated by an actuation mechanism comprising an actuation rod 15, at the end of which this head 13 is mounted, this rod 15 being coupled to an internal housing of the main body of the coring device 10.

This main body here has two body parts 11, 12 engaged one inside the other, a first body part being movable in translation relative to the other body part, such that its movement in and out of the other body part causes the translation movement of said coring head 13.

Each body part has a gripping handle 16, 17 having a longitudinal axis Z. These handles 16, 17 are here arranged in such a way that their longitudinal axis is perpendicular to the longitudinal axis defined by the body of the coring device 10.

Thus, the practitioner can hold with one hand a handle 16 of a first body part 11, while his other hand holds the handle 17 of the other body part 12, in order to easily move these two body parts relative to each other and thus cause the movement of the coring head 13.

Advantageously, the geometric axis or coring axis of the coring device 10 is that of the actuation rod 15 on which the coring head 13 is centered.

The actuation mechanism also comprises a guide carriage 18 supporting the actuation rod 15 and received in the internal housing 19 of the main body. The actuation rod 15 is advantageously centered.

With the guide carriage 18 being rigidly connected to the first body part movable in translation, this internal housing 19 defines a guide rail for translation of this guide carriage.

A lumen 20 extends through the proximal end of the main body of the coring device 10, the actuation rod 15 and the coring head 13 in order to allow the passage of a guidewire (not shown). This wire is intended for guiding, in particular aligning, the coring head 13 as the latter approaches the ventricular wall in order to form an opening by coring.

Advantageously, the distal end of the main body of the coring device 10 also forms an accessory support 21.

This accessory support 21 has a tubular outer peripheral wall whose outer diameter is equal to the diameter of the transection blade 14.

An assistance accessory 22, for assisting in the placement of a part of an anchoring ring of a heart, is received by positive engagement on this accessory support 21. This assistance accessory 22 is hollow and has a sheath extending from an intermediate portion thereof toward its distal end.

As is shown in FIG. 4, while the coring head 13 is still partially placed in the ventricular chamber, the practitioner can insert, into the opening made in the ventricular wall, a part of the assistance accessory 22, in particular its sheath 23, by moving it in translation on the accessory support 21, which ensures linear guiding.

With this assistance tool being made of a rigid material such as titanium, its sheath 23 maintains the opening at its maximum extension and, with its internal channel, delimits a path for the free passage of an anchoring ring part comprising a tubular portion and, at the distal end of the latter, a self-expanding membrane 24 in its first configuration, i.e. a deformed configuration, in which it has a tubular or substantially tubular shape. This self-expanding membrane 24 is made of Nitinol, for example.

Another flange, or second flange 25, of the anchoring ring having been previously connected, for example by suturing, to the outer surface of the ventricular wall, the assistance accessory 22 has, at its proximal end, a hollow cylindrical connection head 26 forming a protrusion of the sheath 23, partially surrounding the latter. This connection head 26 is intended to be connected to this second flange 25 in order to achieve a removable coupling of the accessory to the assembly composed of the second flange and the apex of the heart. This second flange 25 is made of Dacron®, for example.

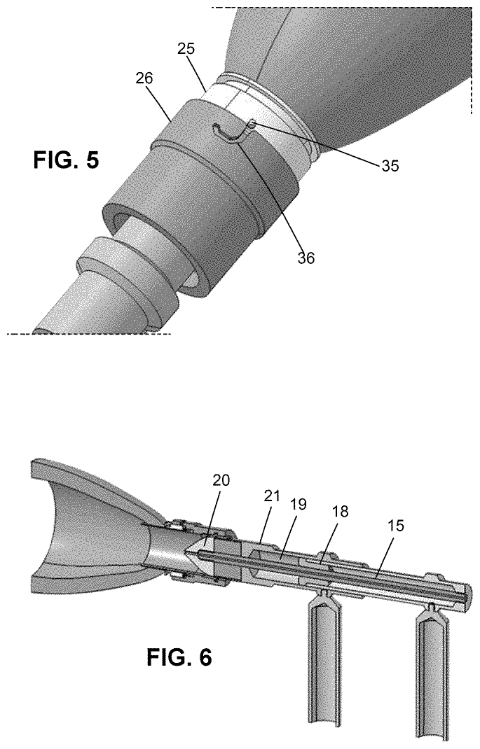

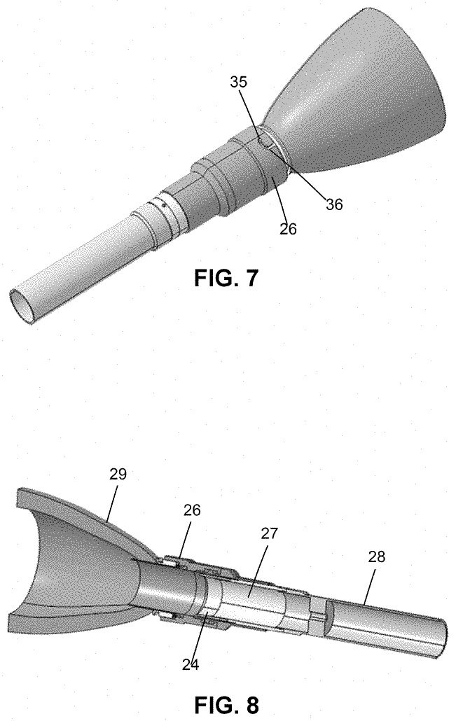

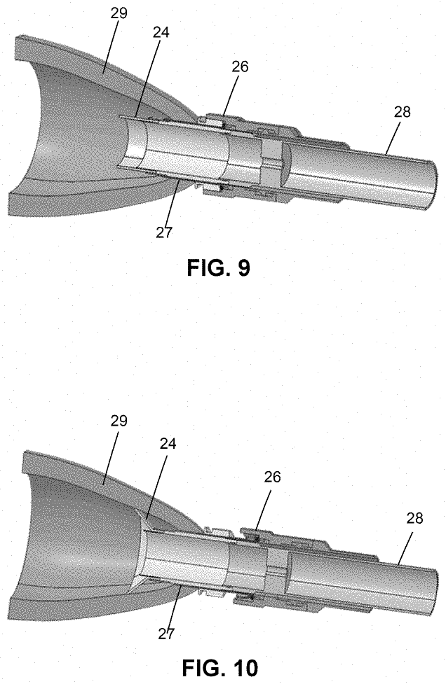

As is illustrated in FIG. 5, with this second flange 25 being pressed against the outer surface of the ventricular wall, it comprises at least one stub 35 positioned on its lateral wall, the connection head 26 of the assistance accessory 22 having, for each stub 35, a coupling groove 36 placed on its edge and configured to receive this corresponding stub. More precisely, each groove 36 opens out at the distal end of this connection head 26, in such a way that, with the latter partially covering the second flange 25 of the anchoring ring, each stub 35 is engaged in its corresponding groove.

Each groove 36 also has the same curved shape so that, by rotation of the assistance accessory 22 around the coring axis, this assistance accessory 22 is locked on the second flange 25.

This assistance accessory 22 also has a sealing element for sealing the assembly composed of connection head 26 and second flange 25, when the one or more stubs have been brought to the bottom of their corresponding groove on the connection head 26.

FIG. 6 is a sectional view of the coring device, the coring head being set back from the opening made in the ventricular wall, and, more precisely, being placed in the internal housing of the body of the coring device. The sheath 23 of the assistance accessory 22 has been inserted through this opening into the ventricular chamber, and the connection head 26 is connected to the second flange 25 of the anchoring ring outside the heart. The practitioner can then withdraw the coring device 10 without risking a displacement of the assistance accessory 22 from the opening made in the ventricular wall.

The sheath 23 of this assistance accessory 22 makes it possible to keep this opening fully accessible in order to ensure the free passage of the anchoring ring part comprising a tubular portion 27, or hollow body, and, at the distal end thereof, a self-expanding membrane 24 in its first configuration, called a deformed configuration.

This withdrawal of the coring device 10 causes the first loss of leaktightness at the ventricular wall, such that the practitioner must quickly connect to this hollow accessory a pusher device 28 carrying this anchoring ring part, tubular portion 27/self-expanding membrane 24, for the insertion of its end into the ventricular chamber 29.

Having inserted the self-expanding membrane 24 into this ventricular chamber, and said membrane having adopted its second configuration in which it defines a flange, or collar, extending radially from the tubular portion 27, the practitioner draws back the pusher device 28 in order to press this flange against the inner face of the ventricular wall 29 of the beating heart.

Thus, with the two flanges 24, 25 being pressed on either side against this ventricular wall 29, the practitioner simply has to engage a sealing element 30 such as an O-ring seal and a clamping means 31 on the pusher device 28 and slide them along the outer surface of this pusher device 28 in order to lock the anchoring ring in position. This clamping means 31 finalizes the assembly of the anchoring ring by blocking any movement of the flanges 24, 25 relative to each other. This results in a strong mechanical connection of the anchoring ring and the apex of the heart and also its sealing.

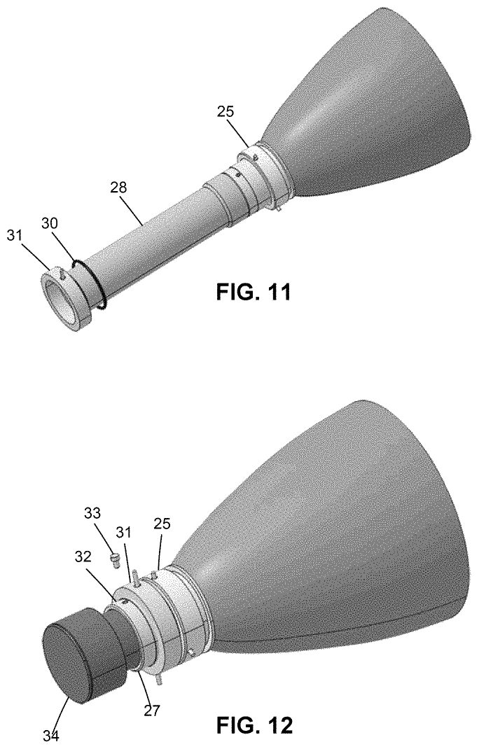

This clamping means 31 can be a nut if the outer surface of the tubular portion 27, or hollow body, has a thread or, as is illustrated in FIG. 12, a clamping ring. The latter presses the sealing element 30 against the second flange 25 connected to the outer surface of the ventricular wall 29.

Advantageously, the assembly composed of tubular portion 27 and self-expanding membrane 24 comprises a second sealing element comprising an anti-reflux valve (not shown) placed in the internal channel delimited by this tubular portion 27.

This clamping ring 31 has, on its lateral wall, an orifice 32 receiving a fastening element 33 for locking in position a heart pump 34 inserted into the internal channel delimited by the hollow body of the anchoring ring, the distal end of this heart pump 34 being engaged in the ventricular chamber of the beating heart.

What is claimed is:

1. A medical device for anchoring a heart pump in an opening of a ventricular wall of a heart, comprising:
a first flange comprising a hole,
an assembly having a hollow body, at the distal end of which is placed a membrane which is self-expandable between a first configuration, called a deformed configuration, in which said membrane has a tubular shape, and a second configuration, called an initial configuration, in which it defines a second flange,
said first flange being separate from said assembly, said assembly being configured to be at least partially introduced through the hole of said first flange, and
a clamping means for connecting said first flange and the proximal end of said hollow body when this assembly has been partially inserted into said hole, said clamping means being configured to slide along said hollow body in such a way as to be pressed against said first flange.

2. The device as claimed in claim 1, characterized in that said first flange is made of a material that can be sutured or stapled.

3. The device as claimed in claim 1, characterized in that said first flange having two end faces connected to each other by a side wall, its end face intended to come into abutment against the outer surface of said ventricular wall, comprises a circular groove in communication with an orifice opening out on its side wall.

4. The device as claimed in claim 1, characterized in that it comprises a first sealing element intended to be placed between said clamping means and said first flange.

5. The device as claimed in claim 1, characterized in that it comprises a second element sealing element comprising an anti-reflux valve placed in the internal channel delimited by said hollow body.

6. The device as claimed in claim 1, characterized in that said hollow body has a length greater than the sum of the thickness of the ventricular wall and said first flange.

7. The device as claimed in claim 1, characterized in that said clamping means is configured to effect a reversible or temporary coupling of said first flange and the proximal end of said hollow body.

8. The device as claimed in claim 1, characterized in that said clamping means is a nut.

9. The device as claimed in claim 8, characterized in that, with this nut having an internal thread, the outer surface of a part of the hollow body, in particular its proximal end, has a complementary thread matching the thread of the nut in order to ensure the engagement and the movement of the nut along said outer surface part of said hollow body.

10. The device as claimed in claim 1, characterized in that the proximal end of the hollow body has a bore for the screwing-in or passage of a fastening element, so as to lock in position a heart pump that is introduced into this hollow body.
